(19) United States
(12) Patent Application Publication
(10) Pub. No.: US 2002/0004533 A1
(43) Pub. Date: Jan. 10, 2002

(54) Integration of shift reactors and hydrotreaters

(75) Inventors: Paul S. Wallace, Katy, TX (US); Kay A. Johnson, Missouri City, TX (US); Cynthia Caputo, Houston, TX (US)

Correspondence Address: STEPHEN H. CAGLE, HOWREY, SIMON, ARNOLD & WHITE, LLP, 750 BERING DRIVE, HOUSTON, TX, 77057, US

(73) Assignee: Texaco Inc.

(21) Appl. No.: 09/773,470

(22) Filed: Jan. 31, 2001

Related U.S. Application Data
(60) Provisional application No. 60/179,507, filed on Feb. 1, 2000.

(51) Int. Cl. C07C 27/06

(52) U.S. Cl. 518/712

(57) ABSTRACT

In this invention, a hydrogen recycle stream from a hydrotreater is heated before returning to the hydrotreater using the energy from a first shift reaction, thereby eliminating the need for a fired heater to heat the hydrogen recycle stream. This heat integration significantly reduces the overall capital and operating costs as well as emissions for the refinery because no fired heater is needed for the hydrotreater and no boiler is needed to cool the effluent from the first stage of shift.

### BACKGROUND OF THE INVENTION

Hydrotreating is an essential process for a refinery in which the catalytic hydrogenation of petroleum is used to release low sulfur liquids and H₂S from sulfur rich hydrocarbons and ammonia from nitrogen containing hydrocarbons to produce reduced sulfur and reduced nitrogen petroleum. Hydrotreaters typically operate at 600-780° F. and use a fired heater to heat the feed stream to the reaction temperature. Oil is fed to the hydrotreater with an excess of hydrogen. The hydrotreater reactor removes sulfur, nitrogen, metals, and coke precursors from the oil. Coking in the fired heater is a significant cause of down time for the hydrotreater because as the oil is heated, localized coking occurs. Coking reduces the efficiency of the fired heater because the buildup of coke on the walls of the heater inhibits the heat transfer. When the flow to the heater becomes too impaired, the process must be taken off line and the coke removed before continuing.

Gasification has been used for years to generate hydrogen gas and fuel gas (also known as synthesis gas or “syn-gas”) from hydrocarbon streams such as coal, petroleum coke, residual oil, and other materials. The hydrocarbon is gasified in the presence of oxygen which is usually generated by an air separation plant in which nitrogen is removed from the air to form the purified oxygen. The availability of hydrogen has led to the use of gasification as a feedstock preparation unit for refinery processes such as hydrotreating units. Synthesis gas from gasification has also been used as a fuel to combustion turbines for the generation of electrical power.

The production of synthesis gas from the solid and liquid carbonaceous fuels, especially coal, coke, and liquid hydrocarbon feeds, has been utilized for a considerable period of time and has recently undergone significant improvements due to the increased energy demand and the need for clean utilization of otherwise low value carbonaceous material. Synthesis gas may be produced by heating carbonaceous fuels with reactive gases, such as air or oxygen, often in the presence of steam or water in a gasification reactor to obtain the synthesis gas which is withdrawn from the gasification reactor.

The synthesis gas may be then further treated, often by separation to form a purified hydrogen gas stream. The synthesis gas stream can be processed to obtain a hydrogen gas stream of greater than 99.9 mole percent purity. The hydrogen gas provides a source for feedstocks for many different refinery processes. For example, the purified H₂ product may be preheated and sent to a hydrotreating unit to produce higher valued petroleum products at a lower cost.

In spite of these and other developments, there exists a continuing need in the industry for an effective method of utilizing the synthesis gas generated by the gasification process.

### SUMMARY OF THE INVENTION

In this invention, the hydrogen recycle stream from the hydrotreater is heated before returning to the hydrotreater using the energy from a first shift reaction, therefore, there is no need for a fired heater to heat the hydrogen recycle stream. Syngas generated from a gasification reactor, containing primarily H2 and CO, is shifted in a first shift reactior to increase the amount of H2 in the gas. The outlet of the first shift reactor provides the heat to the hydrogen recycle stream, and after further treating is usually fed to the hydrotreater as well. This heat integration significantly reduces the overall capital and operating costs as well as emissions for the refinery because no fired heater is needed for the hydrotreater and no boiler is needed to cool the effluent from the first stage of shift.

The effluent from the final stage of the shift reaction must be cooled to allow downstream CO₂ removal. For the hydrogen to be used in the hydrotreater, the CO₂ must also be removed. Physical solvents such as Selexol and Rectisol operating at ambient or refrigerated temperatures are the most common method used for removal of acid gases such as CO₂. Heat from the final stage shift reactor may be used to reheat the hydrogen after CO₂ removal and the CO₂ stream removed from the hydrogen. By doing so the heat duties are balanced because both the hydrogen and CO₂ streams are reheated.

The solvent removes the CO₂ from the hydrogen. The solvent is stripped with nitrogen to remove the CO₂ so that the solvent can be recycled in the acid gas removal process. The stripping liberates a stream that is predominantly CO₂ and nitrogen. This stream is typically routed to a combustion turbine to be used as a fuel diluent.

In order to use the hydrogen in the hydrotreater, residual CO and CO₂ must be converted to methane. The methanation step usually requires a steam preheat of the H₂ rich stream for the reaction to take place, but with this exchanger configuration no external heating is needed to prepare the H₂ stream for the methanator.

The invention uses heat exchangers to produce heated hydrogen for the hydrotreater. The energy from the exothermic shift and methanation reactors is used to saturate the feed gas and heat the product hydrogen and CO₂ diluent streams. The result of these heat exchanger configurations is a reduction in the overall capital and operating costs because no fired heaters or boilers are required to control the heat balance during startup and operation.

The invention may be employed at any site where gasification is used to make hydrogen for refining processes and fuel for combustion turbines.

Some of the advantages of the present invention which should be apparent to one of skill in the art include:

The energy from the effluent stream of the first stage shift reactor is exchanged to heat the sweet H₂ recycle for the hydrotreater.

No fired heater is needed to heat the recycle H₂ from the hydrotreater, which decreases operating and capital costs, increases safety, and decreases emissions. In a hydrotreater, the energy required to start the reaction is usually added to the oil fed to the unit to be hydrotreated, because the oil is usually easier and safer to heat than H2 in a fired heater. Since the current invention uses process heat to heat the H2, it is safer to heat the H2, and the efficiency of the exchange is not an issue since waste heat being used for the exchange that would otherwise not be used.

The hydrotreater is more efficient because no fuel consumption.

Better yield can be achieved from hydrotreater due to increased run time because no coking in the fired heater.

No startup preheater is needed for the methanator because feed/effluent exchanger is used around the upstream CO₂ removal unit.

The risk of contamination of the H₂ stream coming out of the solvent unit is minimized because syngas, mostly H₂ and CO₂, is directly heating the H₂-rich stream. If any CO₂ gets into H₂-rich stream, it will react to form CH₄ in the methanator.

The diluent CO₂ is preheated before entering the combustion turbine, which increases its efficiency.

These and other features of the present invention should be apparent to one of skill in the art in view of the present disclosure.

### BRIEF DESCRIPTION OF THE DRAWINGS

The following description is presented with reference to the accompanying drawings in which:

FIG. 1 is a schematic of an illustrative embodiment of the present invention in which a sweet hydrogen feed is passed into the shift reactors.

FIG. 2 shows a schematic of the hydrotreator unit portion of the embodiment shown in FIG. 1, and is also used with the embodiment shown in FIG. 3.

FIG. 3 provides an overview of an illustrative embodiment of the present invention in which a sour hydrogen feed is passed into the shift reactors.

FIG. 4 is a flow diagram illustrating the general design flow and generalized components of two different embodiments of the present invention.

### DESCRIPTION OF ILLUSTRATIVE EMBODIMENTS

Gasification

Hydrocarbonaceous materials may be gasified to create a mixture of hydrogen, carbon monoxide and carbon dioxide also known as synthesis gas. The gasification and subsequent combustion of certain hydrocarbonaceous materials provides an environmentally friendly method of generating power and desired chemical feedstocks from these otherwise environmentally unfriendly materials. The term “hydrocarbonaceous” as used herein to describe various suitable feedstocks is intended to include gaseous, liquid, and solid hydrocarbons, carbonaceous materials, and mixtures thereof. In fact, substantially any combustible carbon-containing organic material, or slurries thereof, may be included within the definition of the term “hydrocarbonaceous”. Solid, gaseous, and liquid feeds may be mixed and used simultaneously; and these may include paraffinic, olefinic, acetylenic, naphthenic, and aromatic compounds in any proportion. Also included within the definition of the term “hydrocarbonaceous” are oxygenated hydrocarbonaceous organic materials including carbohydrates, cellulosic materials, aldehydes, organic acids, alcohols, ketones, oxygenated fuel oil, waste liquids and by-products from chemical processes containing oxygenated hydrocarbonaceous organic materials, and mixtures thereof. Coal, petroleum based feedstocks including petroleum coke and other carbonaceous materials, waste hydrocarbons, residual oils and byproducts from heavy crude oil are commonly used for gasification reactions.

The hydrocarbonaceous fuels are reacted with a reactive oxygen-containing gas, such as air, or substantially pure oxygen having greater than about 90 mole percent oxygen, or oxygen enriched air having greater than about 21 mole percent oxygen. Substantially pure oxygen is preferred. To obtain substantially pure oxygen, air is compressed and then separated into substantially pure oxygen and substantially pure nitrogen in an oxygen plant. Such oxygen plants are known in the industry.

Synthesis gas can be manufactured by any partial oxidation method. Preferably, the gasification process utilizes substantially pure oxygen with above about 95 mole percent oxygen. The gasification processes are known to the art. See, for example, U.S. Pat. No. 4,099,382 and U.S. Pat. No. 4,178,758, the disclosures of which are incorporated herein by reference.

In the gasification reactor, the hydrocarbonaceous fuel is reacted with a free-oxygen containing gas, optionally in the presence of a temperature moderator, such as steam, to produce synthesis gas. In the reaction zone, the contents will commonly reach temperatures in the range of about 900° C. to 1700° C., and more typically in the range of about 1100° C. to about 1500° C. Pressure will typically be in the range of about 1 atmosphere (101 kPa) to about 250 atmospheres (25,250 kPa), and more typically in the range of about 15 atmospheres (1,515 kPa) to about 150 atmospheres (15,150 kPa), and even more typically in the range of about 800 psi (5,515 kPa) to about 2000 psi (13,788 kPa) (where: 1 atmosphere=101.325 kPa and 1 psi=6.894 kPa).

Synthesis gas predominately includes carbon monoxide gas and hydrogen gas. Other materials often found in the synthesis gas include hydrogen sulfide, carbon dioxide, ammonia, hydrocarbons, cyanides, and particulates in the form of carbon and trace metals. The extent of the contaminants in the synthesis gas is determined by the type of feed, the particular gasification process utilized and the operating conditions.

As the synthesis gas is discharged from the gasifier, it is usually subjected to a cooling and cleaning operation involving a scrubbing technique wherein the gas is introduced into a scrubber and contacted with a water spray which cools the gas and removes particulates and ionic constituents from the synthesis gas. The cooling may be accompanied by heat recovery in the form of high and low pressure steam generation, but also beneficially by heat extraction using heat exchangers wherein low level heat is used to preheat reactants, or to vaporize nitrogen from the oxygen plant.

Desulfurization and Gas Separation

The initially cooled synthesis gas may be treated to desulfurize the synthesis gas prior to utilization. Sulfur compounds and acid gases can be readily removed by use of convention acid gas removal techniques. Solvent fluids containing amines, such as MDEA, can be used to remove the most common acid gas, hydrogen sulfide, but also other acid gases. The fluids may be lower monohydric alcohols, such as methanol, or polyhydric alcohols such as ethylene glycol and the like. The fluid may also contain an amine such as diethanolamine, methanol, N-methyl-pyrrolidone, or a dimethyl ether of polyethylene glycol. Physical solvents such as SELEXOL and RECTISOL may also be used. The physical solvents are typically used because they operate better at high pressure. The synthesis gas is contacted with the physical solvent in an acid gas removal contactor which may be of any type known to the art, including trays or a packed column. Operation of such an acid removal contactor should be known to one of skill in the art.

The synthesis gas may beneficially be subjected to the water-gas shift reaction in the presence of steam (i.e. steam shifted) to increase the fraction of hydrogen. In one embodiment, the synthesis gas is steam shifted to increase the fraction of hydrogen prior to separation, then a hydrogen-rich fraction of the synthesis gas is separated from the shifted synthesis gas. In another embodiment, a hydrogen-rich fraction of the synthesis gas is steam shifted after it is separated from the sulfur and acid gas. In yet another embodiment, the synthesis gas is steam shifted to increase the fraction of hydrogen prior to separation, then a hydrogen-rich fraction of the synthesis gas is separated, and then the separated hydrogen-rich fraction is steam shifted additional times to increase the fraction of recovered hydrogen.

The synthesis gas can be separated with a gas separation membrane into a hydrogen-rich gas and a hydrogen-depleted gas. A gas separation membrane system allows small molecules like hydrogen to selectively pass through the membrane (permeate) while the larger molecules (CO₂, CO) do not pass through the membrane (no-permeate). Gas separation membranes are a cost effective alternative to a pressure swing absorption unit. The gas separation membranes reduce the pressure of the product hydrogen so that the hydrogen rich fraction has to be compressed prior to use.

The gas separation membrane can be of any type which is preferential for permeation of hydrogen gas over carbon dioxide and carbon monoxide. Many types of membrane materials are known in the art which are highly preferential for diffusion of hydrogen compared to nitrogen, carbon monoxide and carbon dioxide. Such membrane materials include: silicon rubber, butyl rubber, polycarbonate, poly(phenylene oxide), nylon 6,6, polystyrenes, polysulfones, polyamides, polyimides, polyethers, polyarylene oxides, polyurethanes, polyesters, and the like. The gas separation membrane units may be of any conventional construction, and a hollow fiber type construction is preferred.

The gas separation membranes cause a reduction in the pressure of the hydrogen-enriched stream so it has to be compressed prior to use. The synthesis gas or mixed gas stream enters the membrane at high pressure, typically between about 800 psi (5,515 kPa) and about 1600 psi (11,030 kPa), more typically between about 800 psi (5,515 kPa) and about 1200 psi (8,273 kPa). The gas temperature is typically between about 10° C. to about 100° C., more typically between about 20° C. and about 50° C. The gas separation membrane allows small molecules like hydrogen to pass through (permeate) while the larger molecule (CO₂, CO) do not pass through (non-permeate). The permeate experiences a substantial pressure drop of between about 500 psi (3,447 kPa) to about 700 psi (4,826 kPa) as it passes through the membrane. The hydrogen-rich permeate is therefore typically at a pressure of from about 100 psi (689 kPa) to about 700 psi (4826 kPa), more typically between about 300 psi (2,068 kPa) to about 600 psi (4,136 kPa).

The hydrogen rich permeate may contain between about 50 to about 98 mole percent hydrogen gas. If the synthesis gas was steam shifted prior to the membrane separation, then the hydrogen content of the permeate, also called the hydrogen-rich synthesis gas, will be at the upper end of this range. If the synthesis gas was not shifted prior to separation, then the hydrogen content of the hydrogen rich permeate will be at the lower end of this range. A typical hydrogen rich permeate composition will be 60 mole percent hydrogen, 20 mole percent carbon monoxide, and 20 mole percent carbon dioxide, plus or minus about 10 mole percent for each component.

The non-permeate has negligible pressure drop in the membrane unit. The non-permeate gas stream from the membrane mostly includes carbon dioxide, carbon monoxide, and some hydrogen. Other compounds, in particular volatile hydrocarbons and inerts, may also be present. It has been found that this non-permeate makes a good fuel for combustion turbines. The pressure of this non-permeate may be advantageously reduced in a turbo-expander to generate electricity or provide energy to compressors prior to burning in a combustion turbine.

The hydrogen stream used for the hydrotreater may need to be compressed to be used in, for example, a high pressure hydrotreater. Such compression can be done at any time. Preferably an expander/compressor combination unit may be used to simultaneously increase the hydrogen pressure and to reduce the pressure of the gas going to the combustion turbine.

Water Gas Shift Reactors

The hydrogen-rich gas from membrane or synthesis gas from the gasifier may be then advantageously shifted with steam to convert the carbon monoxide in the synthesis gas to carbon dioxide and hydrogen by way of the water gas shift reaction. One advantage of doing the water gas shift reaction is the removal of carbon monoxide which is a poison for most H₂ consuming processes. The synthesis gas from the gasifier or H₂ rich gas from the gas separation unit is shifted using steam and a suitable catalyst to form hydrogen as shown below.

H₂O+CO=>H₂+CO₂

The shift process, also called a water gas shift process or steam reforming, converts water and carbon monoxide to hydrogen and carbon dioxide. The shift process is described in, for example, U.S. Pat. No. 5,472,986, the disclosure of which is incorporated herein by reference. Steam reforming is a process of adding water, or using water contained in the gas, and reacting the resulting gas mixture adiabatically over a steam reforming catalyst. The advantages of steam reforming are both an increase the amount of hydrogen and a reduction in the carbon monoxide in the gas mixture.

The steam reforming catalyst can be one or more Group VIII metals on a heat resistant support. Conventional random packed ceramic supported catalyst pieces, as used for example in secondary reformers, can be used but, since these apply a significant pressure drop to the gas, it is often advantageous to use a monolithic catalyst having through-passages generally parallel to the direction of reactants flow.

The shift reaction is reversible, and lower temperatures favor hydrogen and carbon dioxide formation. However, the reaction rate is slow at low temperatures. Therefore, it is often advantageous to have high temperature and low temperature shift reactions in sequence. The gas temperature in a high temperature shift reaction typically is in the range 350° C. to 1050° C. High temperature catalysts are often iron oxide combined with lesser amounts of chromium oxide. A preferred shift reaction is a sour shift, where there is almost no methane and the shift reaction is exothermic. Low temperature shift reactors have gas temperatures in the range of about 150° C. to 300° C., more typically between about 200° C. to 250° C. Low temperature shift catalysts are typically copper oxides that may be supported on zinc oxide and alumina. Steam shifting often is accompanied by efficient heat utilization using, for example, product/reactant heat exchangers or steam generators. Such shift reactors are known to the art.

It is preferred that the design and operation of the shift reactor result in a minimum of pressure drop. Thus, the pressure of the synthesis gas is preserved. Generally a series of shift reactors is implemented to reach the desired conversion to hydrogen. This invention can be applied to a series of 1 to 4 shift reactors, but more often 2-3 shift reactors.

Acid Gas Scrubbing

The effluent from the shift reactor or reactors may contain 4 to 50 mole percent carbon dioxide and thus the carbon dioxide content needs to be reduced. The carbon dioxide may be removed from the synthesis gas by contacting the synthesis gas with a suitable solvent in an acid gas removal contactor. Such a contactor may be of any type known to the art, including trays or a packed column. Operation of such an acid removal contactor is known in the art.

The type of fluid that reacts with the acid gas is not important. Thus in the carbon dioxide removal step, so-called “chemical” solvents can be used, such as ethanolamines or potassium carbonate, especially in the established processes such as “Amine Guard”, “Benfield”, “Benfield-DEA”, “Vetrocoke” and “Catacarb”, at any of the pressures contemplated for the process of the process of the invention. Physical solvents may also be used to remove the acid gas content of the synthesis gas. As examples of physical solvents there may be mentioned: tetramethylene sulfone (“Sulfinor’); propylene carbonate (Fluor); N-methyl-2-pyrrolidone (“Purisol”); polyethyleneglycol dimethyl ether (“Selexol”); and methanol (“Rectisol”). Water can also be used, especially if there is pH control of the water. One such method is a carbonate-based water system wherein carbonates such as potassium carbonate in the water lowers the pH. This low pH water absorbs carbon dioxide to form bicarbonate salts. Later, heating this water liberates carbon dioxide and regenerates the potassium carbonate.

The above noted physical solvents are typically used because they operate better at high pressure. For effective use of physical solvents the process pressure is preferably at least 20 bars (2,000 kPa) (1 bar=100 kPa).

The synthesis gas is contacted with the solvent in an acid gas removal contactor. Said contactor may be of any type known to the art, including trays or a packed column. Operation of such an acid removal contactor should be known to one of skill in the art.

Methanation Reactor

Methanation reactions combine hydrogen with residual carbon oxides to form methane and water. These reactions are strongly exothermic and the heat generated from such reactions may be captured and used to generate steam if desired. The catalyst for the methanation is typically nickel supported on a refractory substance such as alumina although other suitable catalysts may be used. The methanation step reduces the carbon oxides to below about 20 ppm, preferably below about 5 ppm. Such methanation reactions and the operation of methanation reactors should be known by one of ordinary skill in the art for example see U.S. Pat. Nos. 3,730,694; 4,151,191; 4,177,202; 4,260,553 or the references cited therein the contents of which are incorporated herein by reference.

The hydrogen resulting from the above described process has a purity of between 90 and about 99.99, more typically between about 95% and 99.9%.

Combustion Turbine

The quality of the fuel gas utilized in the combustion turbine is not adversely affected by the addition of the purge gas, and valuable power generation can be achieved from the combustion of this purge gas in a combustion turbine. The combustion turbine adds air and combusts the mixture, and then the exhaust gases are expanded thorough a turbine. Such combustion turbines are known to the art.

Most gas combustion turbines have lower limits on the amount of heating value per cubic foot of fuel gas. For general use the fuel with the highest heating value is methane, which has, a fuel values of around 900 to 1000 BTU/scf. Other gaseous fuels may have less heating value, down to 300 to 500 BTU/scf, and these can be treated in a somewhat similar manner as natural gas. When, however, the heating value falls below the level of about 300 BTU/scf, a rigorous inspection of gas turbine conditions is called for, this to avoid feeding too much inert material to the expander side.

If the fuel gas has a heating value below about 100 BTU/scf, other problems arise, such as flame stability—the fire in the gas turbine will go out. At this low value it becomes necessary to determine if the fuel gas can be completely burned in the residence time in the burner or burners of the gas turbine before entering the expander proper. Incomplete combustion can lead to deposition of carbonaceous material on the expander blades, which will lead to an early demise of the gas turbine involved. Thus it is essential that the heating value of the tail gas fuel not be too low, preferably it should be at least about 100 BTU/scf. Also, such low BTU/scf fuel gases should have fast burning characteristics. This is especially true when the available burner space of the gas turbine is limited, which in a relatively large number of commercially available gas turbines is indeed the case.

The fastest burning material is hydrogen. A considerable fraction of the heating value of such fuel gas with very low heating value has to be provided by hydrogen. A reasonable fraction is about 30 to 40% as a minimum of the heat of combustion BTU content is supplied by hydrogen. The fast burning hydrogen elevates the temperature of the flame considerably in relatively little space and provides flame stability, whereupon the other combustibles of the low heating value fuel have a greater chance to be burned properly. This may be especially the case when hydrogen has been burned already, and the gas temperature has therefore been increased and hot steam has become available, any CO present in the tail gas fuel will then burn with great speed.

Generally methane present in the fuel gas burns slow. Therefore it is important that the temperature be elevated so that this slow burning species can be totally combusted. Hence it is not attractive to have more than say 30% of total heat of combustion content available as methane in the tail gas fuel.

Illustrative Embodiments

With reference to the figures, the following table provides a key to the reference number and letters shown:

TABLE 1

| Reference | |
|---|---|
| number/letter | Description |
| A | Sour hydrogen from hydrotreater unit (HTU) to H₂S |
| | removal (2) |
| B | Saturator water to low temperature cooling gas unit (LTCG) |
| C | Fuel gas to combustion turbine (CT) |
| D | Sour synthesis gas from gasifier |
| E | Sour fuel gas from hydrotreater unit (HTU) to H₂S |
| | removal (2) |
| G | Nitrogen from air separation unit |
| H | Acid gas to sulfur removal system (SRS) |
| J | High pressure steam to saturator |
| K | Make-up water for saturator |
| L | Saturator water from low temperature cooling gas unit |
| M | CO₂/N₂ Dilution gas to combustion turbine |
| N | Hot recycle H2 to hydrotreater |
| P | Cold recycle hydrogen to hydrotreater |
| Q | Sour oil feed to hydrotreater |
| R | Sweet feed for catalytic hydrocracker |
| S | Light hydrocarbon distillates |
| T | Sour recycle water to gasifier. |
| 2 | H₂S scrubber & gas separator |
| 4 | Seam Heater |
| 6 | Zinc oxide guard bed |
| 8 | Saturator |
| 10 | High pressure steam heater |
| 12 | 1^{st} stage shift reactor |
| 14 | Hydrogen gas heat exchanger |
| 16 | Saturator water preheating heat exchanger |
| 18 | 2^{nd Stage shift reactor} |
| 20 | Gas feed pre-heaters (heat exchangers) |
| 22 | Air cooled heat exchanger |
| 24 | Knockdown drum |
| 26 | Acid gas scrubber (Selexol)/gas separator unit |
| 28 | Methanization reactor |
| 30 | Pre-heat for make-up hydrogen (heat exchanger) |
| 32 | Water cooled heat exchanger |
| 34 | Knockdown drum |
| 36 | Make-up hydrogen compressor |
| 38 | Pressurized hydrogen from H₂S scrubber (optional) |
| 40 | Pump for knockdown drum blowdown water |
| 42 | Pump for knockdown drum blowdown water |
| 44 | Pump for saturator blowdown water |
| 100 | Hydrotreater unit (HTU) |
| 102 | Feed oil preheater |
| 104 | Start-up feed oil preheater (optional) |
| 106 | Knockdown separator |
| 108 | Stripper unit |
| 110 | Air cooled heat exchanger |
| 112 | Water cooled heat exchanger |
| 114 | Sour water separator |
| 116 | Pump for sour water |
| 118 | Water cooled heat exchanger |
| 120 | Hydrocarbon separation drum |
| 122 | Pump for light distillates |
| 200 | Gasifier unit |
| 202 | Wet synthesis gas |
| 204 | Oxygen feed gas |
| 206 | Hydrocarbon feed |
| 208 | H₂S gas removal unit |
| 210 | Sweet hydrogen water gas shift reactor unit |
| 212 | Hydrotreater unit (HTU) |
| 214 | Hydrotreated petroleum |
| 216 | Hydrogen recycle loop |
| 218 | Sour water gas shift reactor unit |
| 220 | H₂S gas removal unit |
| 222 | hydrotreater unit (HTU) |
| 224 | Hydrotreated petroleum |
| 226 | Hydrogen recycle loop. |

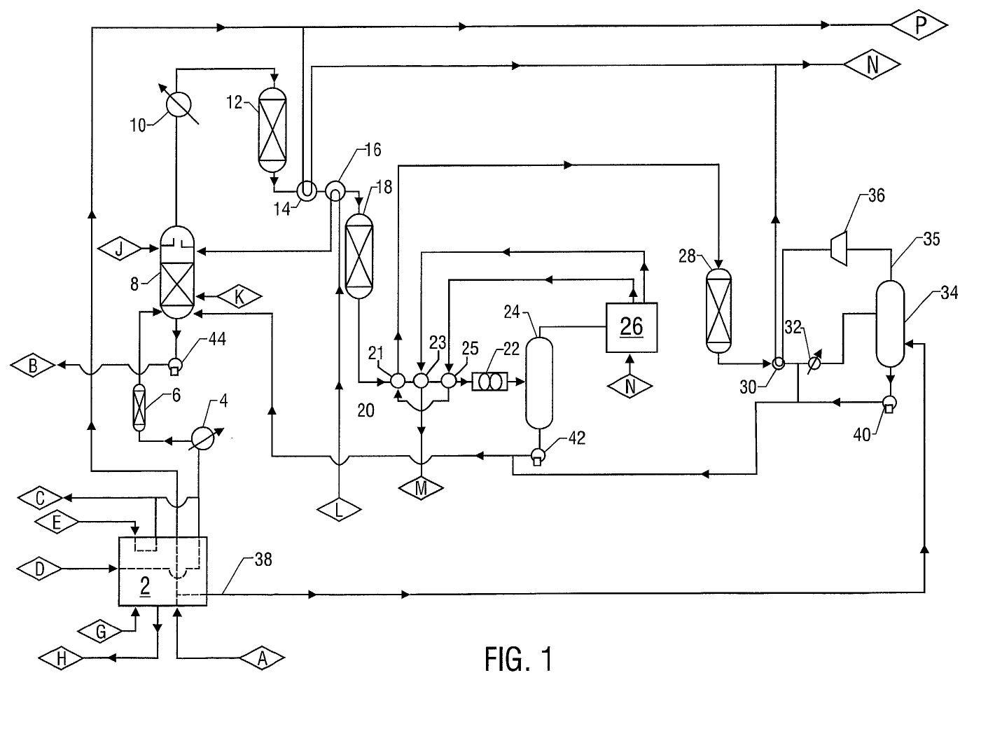

Turning now to FIG. 1, a schematic flow diagram for a sweet water gas shift layout is illustrated. The primary feature of such a layout is that the sour gas component of the syngas is removed prior to sending the hydrogen and carbon monoxide containing gas mixture to the water gas shift reactors. The primary input of gas is sour synthesis gas ‘D’ from the gasifier. After being shifted and purified, the hydrogen from the syngas combines with recycle hydrogen from the hydrotreater to provide a steady source of high pressure and preheated hydrogen gas to the hydrotreater. This is beneficial to using “over the fence” hydrogen that must be heated and compressed prior to introduction into the hydrotreater. By utilizing the gasification reactor as the hydrogen source, the fired heater for the hydrogen is eliminated, thus reducing capital and operating costs and emissions.

As shown in FIG. 1, the H₂S gas scrubber and separator system 2 provides a sweetens a stream of sour synthesis gas ‘D’ that then passes through a steam heater 4 and a zinc oxide guard column 6 prior to being introduced to a water saturator column 8. The saturated gas mixture then passes through high pressure steam heater 10 on its way to the first water gas shift reactor 12. The heat generated by the first water gas shift reactor 12 is utilized to heat recycle hydrogen gas ‘A’ from the hydrotreater in heat exchanger 14 and also to preheat the water being sent to the saturator column 8 in heat exchanger 16. The somewhat cooled gas is then passed through a second water gas shift reactor 18 to further increase the hydrogen content of the gas. The hot gas from the second shift reactor is passed through a series of three exchange loops so that the heat can be recovered. This heat is used to preheat the feed to the methanizer reactor (exchangers 21 and 25) and to heat the CO₂/N₂ diluent ‘M’ from the acid gas scrubber 26 that is being sent to a combustion turbine for power production. An air-cooled heat exchanger 22 further cools the hot gas, which then enters a knockdown drum 24 for separation of the water component from the gas component. The gas component, which is a mixture of hydrogen and carbon dioxide, is then sent to the acid gas scrubber/separator unit 26 so as to remove the CO₂, nitrogen, and other acid gases and to produce a hydrogen-rich stream. The nitrogen and carbon dioxide components of the acid gas scrubber are recovered and sent to a combustion turbine as a diluent ‘M’.

The hydrogen-rich stream is then reheated using heat from the second water gas shift reactor 18 in heat exchangers 25 and 21, and sent to the methanization reactor 28. After the methanization reactor 28, the hot product gas, which contains hydrogen and methane gas, is passed through a heat exchanger 30 to remove heat and condense any water present in the gas. The stream is then passed through a water cooled heat exchanger 32 for further cooling. The gas mixture is then sent to a knockdown drum 34 to remove the condensed water from the gas. The overhead effluent 35, which is primarily hydrogen but also may contain small amounts of methane and inert gasses, is repressurized using hydrogen compressor 36 and reheated using the heat from the methanization reactor 28 outlet stream in heat exchanger 30. This hydrogen stream is then combined with the hydrogen recycle stream recovered in the H₂S separator and heated by the first water gas shift reactor 12 outlet stream, and the combination is then sent to the hydrotreater as hot hydrogen gas ‘N’. A second fraction of the hydrogen recovered by the H₂S scrubber is not reheated by the first water gas shift reactor 12 outlet stream and is instead sent to the hydrotreater as cold hydrogen gas ‘P’ which is used to quench the hydrotreating reaction.

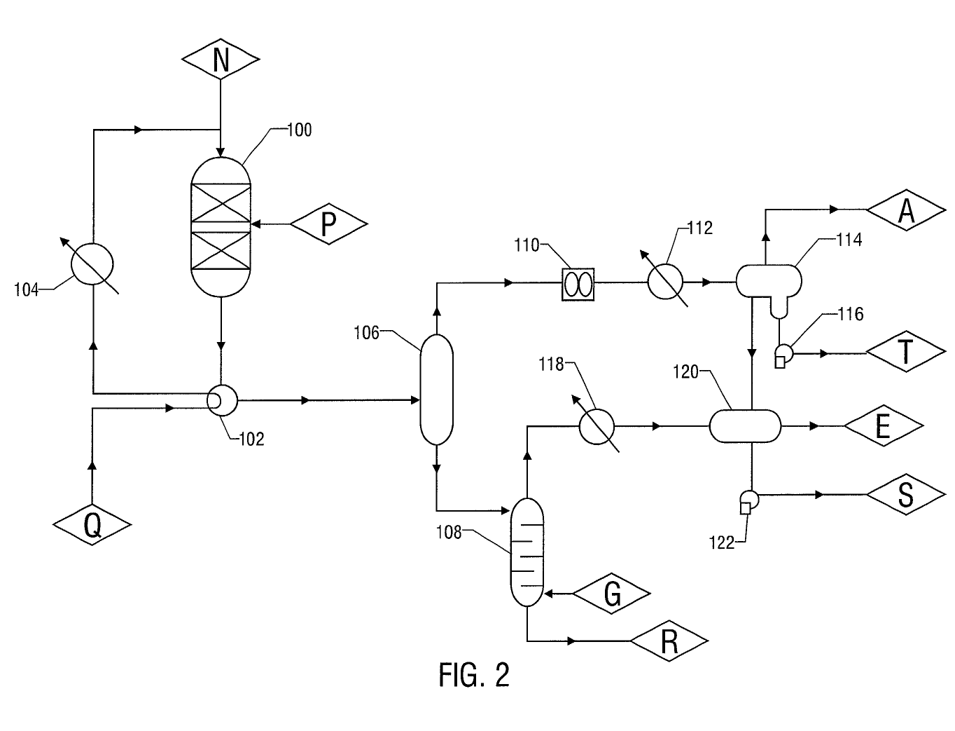

FIG. 2 illustrates an example of the hydrotreating unit section that is integrated with the hydrogen generation scheme just described and as shown in FIG. 1. When used with the reference Table 1, one of ordinary skill in the art will see that in all aspects it is conventional in design.

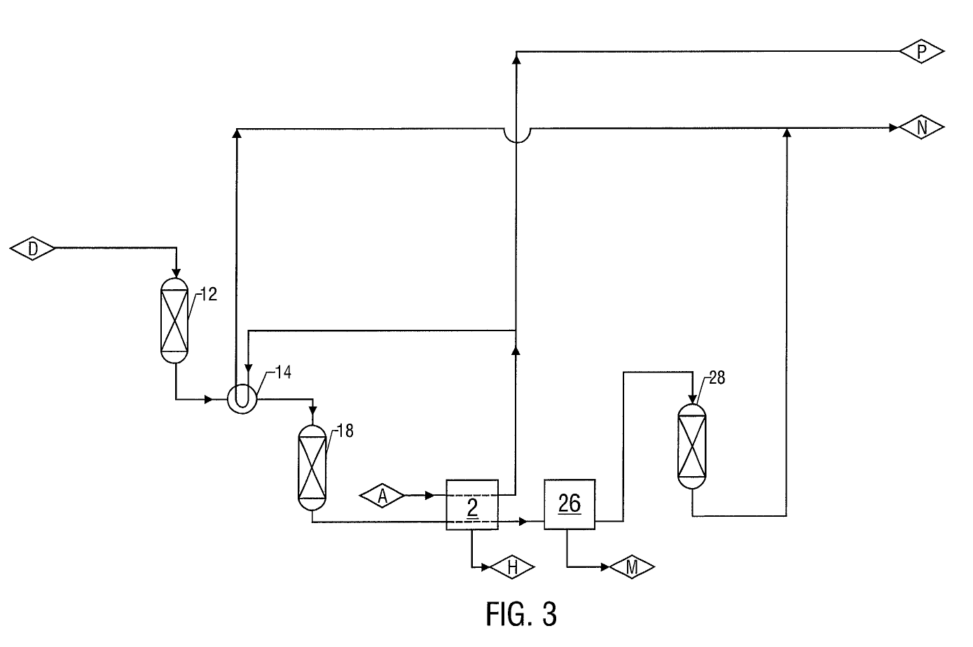

FIG. 3 illustrates the basic components and basic concept of the sour shift reactor embodiment of the present invention. Matching equipment numbers from FIG. 1 are used for the ease and understanding of the drawing. In FIG. 3,

As shown in FIG. 1, a stream of sour synthesis gas ‘D’ is sent to the first water gas shift reactor 12. The heat generated by the first water gas shift reactor 12 is utilized to heat recycle hydrogen gas ‘A’ from the hydrotreater in heat exchanger. The somewhat cooled gas is then passed through a second water gas shift reactor 18 to further increase the hydrogen content of the gas. The hot gas from the second shift reactor is then passed through a H₂S gas scrubber and separator system 2 as well as an acid gas scrubber 26 so that a hydrogen-rich stream is produced. The hydrogen-rich stream is then sent to the methanization reactor 28, producing a hot stream of primarily hydrogen, but also may contain small amounts of methane and inert gasses. This hydrogen stream is then combined with the hydrogen recycle stream recovered in the H₂S separator and heated by the first water gas shift reactor 12 outlet stream, and the combination is then sent to the hydrotreater as hot hydrogen gas ‘N’. A second fraction of the hydrogen recovered by the H₂S scrubber is not reheated by the first water gas shift reactor 12 outlet stream and is instead sent to the hydrotreater as cold hydrogen gas ‘P’ which is used to quench the hydrotreating reaction. FIG. 2, illustrating the example of a hydrotreating unit section, can also be integrated with the sour hydrogen generation process just described and shown in FIG. 3.

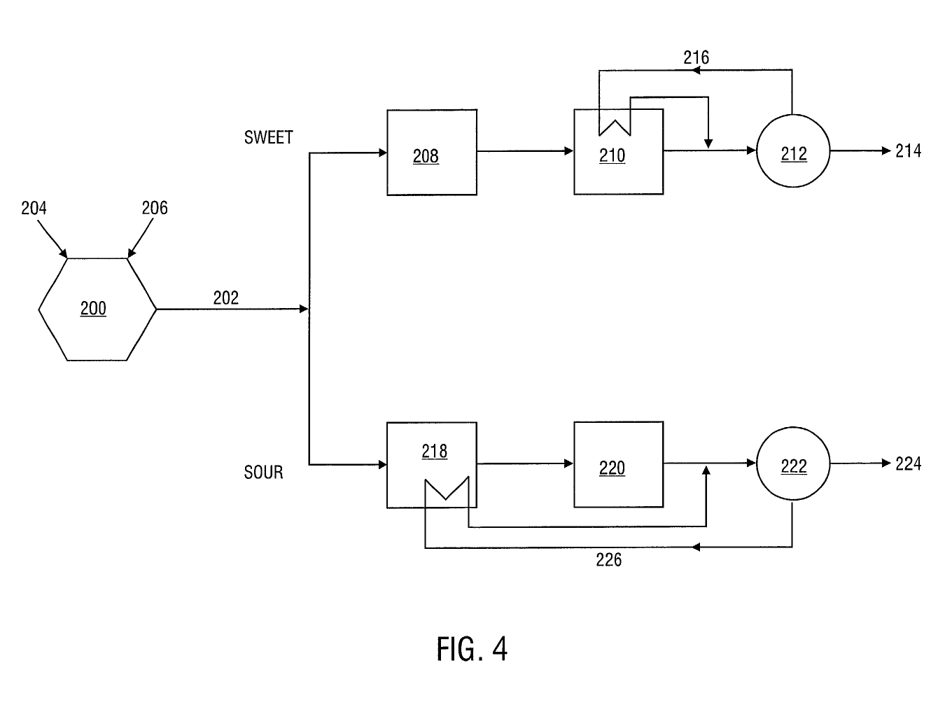

FIG. 4 illustrates the overall concept, relationship and design options of the two illustrative embodiments of the present invention. The gasification unit 200 generates synthesis gas 202 by the controlled oxidation of hydrocarbon feed 204 in the presence of an oxygen feed 206. The synthesis gas may be utilized in a sweet shift reactor layout as illustrated in FIGS. 1 and 2, or in a sour shift reactor layout as illustrated in FIGS. 3 and 2.

Generally the sweet shift reactor layout has an H₂S gas removal unit 208 prior to a sweet hydrogen water gas shift reactor unit 210, which may consist of one or more water gas shift reactors. The product hydrogen gas is utilized in the hydrotreating unit 212 to give hydrotreated petroleum 214. A recycle loop 216 for the hydrogen gas not consumed in the hydrotreating process is provided and exchanges heat with an outlet of a water gas shift reactor unit.

In contrast the sour shift reactor layout has a sour hydrogen gas water gas shift reactor unit 218 prior to an H₂S gas removal unit 220. The product hydrogen gas is utilized in the hydrotreating unit 222 to give hydrotreated petroleum 224. A recycle loop 226 for the hydrogen gas not consumed in the hydrotreating process is provided and exchanges heat with an outlet of a water gas shift reactor unit.

The selection of either a sweet shift reactor layout or a sour shift reactor layout will depend upon a number of factors including the carbonaceous feed to the gasifier, the H₂S gas content of the synthesis gas, the availability and capacity of existing facilities, and other factors which should be apparent to one of skill in the art. Other details regarding the present illustrative embodiments will be apparent to one of skill in the art and as such are considered to be within the scope of the present invention.

The above illustrative embodiments are intended to serve as simplified schematic diagrams of potential embodiments of the present invention. One of ordinary skill in the art of chemical engineering should understand and appreciate that specific details of any particular embodiment may be different and will depend upon the location and needs of the system under consideration. All such layouts, schematic alternatives, and embodiments capable of achieving the present invention are considered to be within the capabilities of a person having skill in the art and thus within the scope of the present invention.

While the apparatus, compounds and methods of this invention have been described in terms of preferred embodiments, it will be apparent to those of skill in the art that variations may be applied to the process described herein without departing from the concept and scope of the invention. All such similar substitutes and modifications apparent to those skilled in the art are deemed to be within the scope and concept of the invention.

What is claimed is:

1. An process for integrating hydrotreating reactors and syngas shift reactors comprising exchanging heat between at least a portion of a recycle stream of hydrogen from a hydrotreating reactor and an outlet stream of a syngas shift reactor.

2. The process of claim 1, wherein the recycle stream of hydrogen is heated to a sufficient temperature to provide reaction initiation energy for processing in the hydrotreating reactor.

3. The process of claim 1, wherein the recycle stream of hydrogen contains sulfur compounds, and prior to exchanging heat with the outlet stream of the shift reactor the sulfur is removed from the recycle stream of hydrogen.

4. The process of claim 1, further comprising a gasification reactor integrated with the hydrotreating reactors and the shift reactors, wherein the gasification reactor produces the hydrogen feed stream to the hydrotreater.

5. The process of claim 4, further comprising treating a synthesis gas product from the gasification reactor so as to remove any sulfur compounds in the synthesis gas, processing the synthesis gas in the shift, purifying the synthesis gas to produce a hydrogen stream, and feeding hydrogen stream to the hydrotreater.

6. The process of claim 5, wherein the by-products of the synthesis gas purification step are processed in a combustion turbine so as to produce power.

7. The process of claim 5, wherein the hydrogen stream is at least 90% pure hydrogen.

8. The process of claim 7, wherein the hydrogen stream is at least 95% pure hydrogen.

9. The process of claim 4, further comprising processing a synthesis gas product from the gasification reactor in the shift reactors, treating the synthesis gas so as to remove any sulfur compounds in the synthesis gas, purifying synthesis gas to produce at least a hydrogen stream, and feeding the hydrogen stream to the hydrotreater.

10. The process of claim 9, wherein the by-products of the synthesis gas purification step are processed in a combustion turbine so as to produce power.

11. The process of claim 9, wherein the hydrogen stream is at least 90% pure hydrogen.

12. The process of claim 11, wherein the hydrogen stream is at least 95% pure hydrogen.

13. A hydrotreating process comprising:
gasifying hydrocarbonaceous materials in a gasification reactor to produce synthesis gas;
processing the synthesis gas in a first shift reactor;
contacting the synthesis gas with a solvent in an acid gas removal contactor, producing a hydrogen stream and an acid gas stream;
reacting the hydrogen stream in a methanation reactor, producing a substantially pure hydrogen stream;
feeding the substantially pure hydrogen stream to a hydrotreating reactor, forming a sour hydrogen recycle stream;
purifying the sour hydrogen recycle stream, forming a sweet hydrogen recycle stream;
exchanging heat between at least a portion of the sweet hydrogen recycle stream and the outlet stream of the first shift reactor;
returning the sweet hydrogen recycle stream to the hydrotreating reactor.

14. The process of claim 13, further comprising desulfurizing the synthesis gas prior to processing the synthesis gas in the first shift reactor.

15. The process of claim 13, wherein the synthesis gas is processed in a plurality of shift reactors.

16. The process of claim 15, wherein the outlet from the last of the plurality of shift reactors exchanges heat with the hydrogen stream and the acid gas stream products of the acid gas removal contactor.

17. The process of claim 13, further comprising desulfurizing the synthesis gas after processing the synthesis gas in the first shift reactor.

18. The process of claim 13 wherein the acid gas stream products of the acid gas removal contactor are burned in a combustion turbine to produce power.
